(19) United States
(12) Patent Application Publication
(10) Pub. No.: US 2022/0313133 A1
(43) Pub. Date: Oct. 6, 2022

(54) OPM MODULE ASSEMBLY WITH ALIGNMENT AND MOUNTING COMPONENTS AS USED IN A VARIETY OF HEADGEAR ARRANGEMENTS

(71) Applicant: HI LLC, Los Angeles, CA (US)

(72) Inventors: Jeffery Kang Gormley, Chatsworth, CA (US); Ethan Pratt, Santa Clara, CA (US); Lucas Marsh, Venice, CA (US); James Coyne, Los Angeles, CA (US); Teresa Giovannoli, Marina Del Rey, CA (US); Dakota Blue Decker, Culver City, CA (US); Stephen Garber, Santa Monica, CA (US); Scott Michael Homan, Culver City, CA (US); Jake Phillips, Marina Del Rey, CA (US)

(21) Appl. No.: 17/338,429

(22) Filed: Jun. 3, 2021

Related U.S. Application Data
(60) Provisional application No. 63/170,892, filed on Apr. 5, 2021.

(51) Int. Cl. A61B 5/245; G01R 33/032; A61B 5/00

(52) U.S. Cl. CPC A61B 5/245; G01R 33/032; A61B 5/6803

(57) ABSTRACT

A headgear for magnetoencephalography includes a body defining a plurality of ports, where the body includes a first portion and a second portion; an adjustment mechanism coupled to the first portion and the second portion of the body and configured to adjust a separation between the first and second portion to facilitate fitting the headgear to a head of a user; and a plurality of optically pumped magnetometer (OPM) modules, where each of the OPM modules includes at least one vapor cell and is configured to be removably inserted into a one of the ports of the body, where each of the OPM modules is configured for coupling to a light source for receiving light.

### CROSS-REFERENCE TO RELATED APPLICATIONS

This application claims the benefit of U.S. Provisional Patent Application Ser. No. 63/170,892, filed Apr. 5, 2021, which is incorporated herein by reference.

### FIELD

The present disclosure is directed to the area of magnetic field measurement systems including systems for magnetoencephalography (MEG). The present disclosure is also directed to optically pumped magnetometer modules and headgear arrangements and methods of making and using.

### BACKGROUND

In the nervous system, neurons propagate signals via action potentials. These are brief electric currents which flow down the length of a neuron causing chemical transmitters to be released at a synapse. The time-varying electrical current within an ensemble of neurons generates a magnetic field. Magnetoencephalography (MEG), the measurement of magnetic fields generated by the brain, is one method for observing these neural signals.

Existing technology for measuring MEG typically utilizes superconducting quantum interference devices (SQUIDs) or collections of discrete optically pumped magnetometers (OPMs). SQUIDs require cryogenic cooling, which is bulky, expensive, requires a lot of maintenance. These requirements preclude their application to mobile or wearable devices.

An alternative to an array of SQUIDs is an array of OPMs. For MEG and other applications, the array of OPMS may have a large number of OPM sensors that are tightly packed. Such dense arrays can produce a high resolution spatial mapping of the magnetic field, and at a very high sensitivity level. Such OPMs sensors can be used for a wide range of applications, including sensing magnetic field generated by neural activities, similar to MEG systems.

### BRIEF SUMMARY

One embodiment is a headgear for magnetoencephalography that includes a body defining a plurality of ports, where the body includes a first portion and a second portion; an adjustment mechanism coupled to the first portion and the second portion of the body and configured to adjust a separation between the first and second portion to facilitate fitting the headgear to a head of a user; and a plurality of optically pumped magnetometer (OPM) modules, where each of the OPM modules includes at least one vapor cell and is configured to be removably inserted into a one of the ports of the body, where each of the OPM modules is configured for coupling to a light source for receiving light.

In at least some embodiments, the plurality of OPM modules is at least two OPM modules with at least one of the OPM modules disposed in the first section and at least another one of the OPM modules disposed in the second section. In at least some embodiments, the plurality of OPM modules is at least ten OPM modules. In at least some embodiments, the headgear further comprises a connection element coupled to the body for coupling the headgear to an arm.

Yet another embodiment is a headgear for magnetoencephalography that includes a body defining a plurality of ports; and a plurality of optically pumped magnetometer (OPM) modules, where each of the OPM modules includes at least one vapor cell and is configured to be removably inserted into a one of the ports of the body, where each of the OPM modules is configured for coupling to a light source for receiving light and the OPM modules include, in total, at least 200 vapor cells.

In at least some embodiments, the OPM modules include, in total, at least 256 vapor cells. In at least some embodiments, each of the OPM modules includes at least six vapor cells.

A further embodiment is a headgear for magnetoencephalography that includes a body defining a plurality of ports; a plurality of optically pumped magnetometer (OPM) modules, where each of the OPM modules includes at least one vapor cell and is configured to be removably inserted into a one of the ports of the body, where each of the OPM modules is configured for coupling to a light source for receiving light; and a plurality of sleeves, where each of the sleeves is configured to fit around a one of the OPM modules and configured to fit, with the OPM module, in a one of the ports and to fix the OPM module within the one of the ports.

In at least some embodiments, each of the sleeves includes at least one snap fit fixture configured to snap fit over a feature on the OMP module. In at least some embodiments, each of the sleeves includes an inner sleeve and an outer sleeve that is movable relative to the inner sleeve. In at least some embodiments, the inner sleeve and the outer sleeve have complementary sets of detents that facilitate positioning the inner sleeve at different depths relative to the outer sleeve by matching the sets of detents.

Another embodiment is a magnetic field measurement system includes any of the headgear disclosed above and at least one laser module coupleable to the OPM modules to provide the laser light.

In at least some embodiments, the magnetic field measurement system further includes at least one sensor module coupleable to the OPM modules to receive output from the detector arrangement. In at least some embodiments, the magnetic field measurement system further includes at least one peripheral configured for input by a user of the magnetic field measurement system while the user wears the headgear.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description, which is to be read in association with the accompanying drawings, wherein:

FIG. 1A is a schematic block diagram of one embodiment of a magnetic field measurement system, according to the invention;

FIG. 1B is a schematic block diagram of one embodiment of a magnetometer, according to the invention;

FIG. 2 shows a magnetic spectrum with lines indicating dynamic ranges of magnetometers operating in different modes;

FIG. 3 is a schematic cross-sectional view of one embodiment of an optically pumped magnetometer (OPM) module, according to the invention;

FIG. 4 is a schematic perspective view of the OPM module of FIG. 3, according to the invention;

FIG. 5 is a schematic perspective view of the OPM module of FIG. 3 disposed in one embodiment of a sleeve, according to the invention;

FIG. 6 is a schematic perspective view of the OPM module of FIG. 3 disposed in a second embodiment of a sleeve, according to the invention;

FIG. 7 is a schematic cross-sectional view of the OPM module of FIG. 3 disposed in the sleeve of FIG. 6, according to the invention;

FIG. 8 is a schematic side view of one embodiment of a headgear with OPM modules of FIG. 3 disposed therein, according to the invention;

FIG. 9 is a schematic top view of the headgear of FIG. 8, according to the invention;

FIG. 10A is a schematic side view of a second embodiment of a headgear with OPM modules of FIG. 3 disposed therein, according to the invention;

FIG. 10B is a schematic top view of the headgear of FIG. 10A, according to the invention;

FIG. 11 is a schematic side view of a third embodiment of a headgear (e.g., a helmet) with OPM modules of FIG. 3 disposed therein, according to the invention;

FIG. 12 is a schematic front view of the headgear of FIG. 11, according to the invention;

FIG. 13 is a schematic perspective view of one embodiment of an arm holding the headgear of FIG. 10A (without the OPM modules of FIG. 3 disposed therein), according to the invention;

FIG. 14 is a schematic perspective view of another embodiment of an arm for holding headgear, according to the invention;

FIG. 15 is a schematic perspective view of one embodiment of a laser module, according to the invention;

FIG. 16 is a schematic perspective view of one embodiment of a sensor module, according to the invention; and

FIG. 17 is a schematic perspective view of one embodiment of a peripheral (e.g., a handheld remote), according to the invention.

### DETAILED DESCRIPTION

The present disclosure is directed to the area of magnetic field measurement systems including systems for magnetoencephalography (MEG). The present disclosure is also directed to optically pumped magnetometer modules and headgear arrangements and methods of making and using.

Herein the terms “ambient background magnetic field” and “background magnetic field” are interchangeable and used to identify the magnetic field or fields associated with sources other than the magnetic field measurement system and the magnetic field sources of interest, such as biological source(s) (for example, neural signals from a user's brain) or non-biological source(s) of interest. The terms can include, for example, the Earth's magnetic field, as well as magnetic fields from magnets, electromagnets, electrical devices, and other signal or field generators in the environment, except for the magnetic field generator(s) that are part of the magnetic field measurement system.

The terms “gas cell”, “vapor cell”, and “vapor gas cell” are used interchangeably herein. Below, a gas cell containing alkali metal vapor is described, but it will be recognized that other gas cells can contain different gases or vapors for operation.

The methods and systems are described herein using optically pumped magnetometers (OPMs). While there are many types of OPMs, in general magnetometers operate in two modalities: vector mode and scalar mode. In vector mode, the OPM can measure one, two, or all three vector components of the magnetic field; while in scalar mode the OPM can measure the total magnitude of the magnetic field.

Vector mode magnetometers measure a specific component of the magnetic field, such as the radial and tangential components of magnetic fields with respect the scalp of the human head. Vector mode OPMs often operate at zero-field and may utilize a spin exchange relaxation free (SERF) mode to reach femto-Tesla sensitivities. A SERF mode OPM is one example of a vector mode OPM, but other vector mode OPMs can be used at higher magnetic fields. These SERF mode magnetometers can have high sensitivity but may not function in the presence of magnetic fields higher than the linewidth of the magnetic resonance of the atoms of about 10 nT, which is much smaller than the magnetic field strength generated by the Earth.

Magnetometers operating in the scalar mode can measure the total magnitude of the magnetic field. (Magnetometers in the vector mode can also be used for magnitude measurements.) Scalar mode OPMs often have lower sensitivity than SERF mode OPMs and are capable of operating in higher magnetic field environments.

The magnetic field measurement systems, such as a MEG system, described herein can be used to measure or observe electromagnetic signals generated by one or more magnetic field sources (for example, biological sources) of interest. The system can measure biologically generated magnetic fields and, at least in some embodiments, can measure biologically generated magnetic fields in a partially shielded environment. Aspects of a magnetic field measurement system will be exemplified below using magnetic signals from the brain of a user; however, biological signals from other areas of the body, as well as non-biological signals, can be measured using the system. In at least some embodiments, the system can be a wearable MEG system that can be portable and used outside a magnetically shielded room. A wearable MEG system will be used to exemplify the magnetic field measurement systems and calibration arrangements described herein; however, it will be recognized the calibration arrangements and methods described herein can be applied to other magnetic field measurement systems.

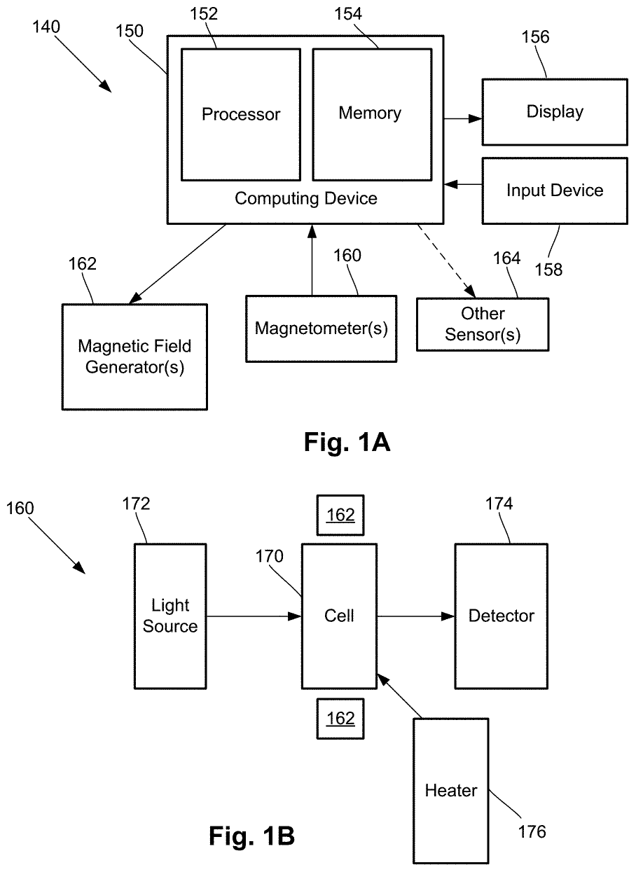

A magnetic field measurement system, such as a MEG system, can utilize one or more magnetic field sensors. Magnetometers will be used herein as an example of magnetic field sensors, but other magnetic field sensors may also be used in addition to, or as an alternative to, the magnetometers. FIG. 1A is a block diagram of components of one embodiment of a magnetic field measurement system 140 (such as a biological signal detection system.) The system 140 can include a computing device 150 or any other similar device that includes a processor 152, a memory 154, a display 156, an input device 158, one or more magnetometers 160 (for example, an array of magnetometers) which can be OPMs, one or more magnetic field generators 162, and, optionally, one or more other sensors 164 (e.g., non-magnetic field sensors). The system 140 and its use and operation will be described herein with respect to the measurement of neural signals arising from one or more magnetic field sources of interest in the brain of a user as an example. It will be understood, however, that the system can be adapted and used to measure signals from other magnetic field sources of interest including, but not limited to, other neural signals, other biological signals, as well as non-biological signals.

The computing device 150 can be a computer, tablet, mobile device, field programmable gate array (FPGA), microcontroller, or any other suitable device for processing information or instructions. The computing device 150 can be local to the user or can include components that are non-local to the user including one or both of the processor 152 or memory 154 (or portions thereof). For example, in at least some embodiments, the user may operate a terminal that is connected to a non-local computing device. In other embodiments, the memory 154 can be non-local to the user.

The computing device 150 can utilize any suitable processor 152 including one or more hardware processors that may be local to the user or non-local to the user or other components of the computing device. The processor 152 is configured to execute instructions stored in the memory 154.

Any suitable memory 154 can be used for the computing device 150. The memory 154 illustrates a type of computer-readable media, namely computer-readable storage media. Computer-readable storage media may include, but is not limited to, volatile, nonvolatile, non-transitory, removable, and non-removable media implemented in any method or technology for storage of information, such as computer readable instructions, data structures, program modules, or other data. Examples of computer-readable storage media include RAM, ROM, EEPROM, flash memory, or other memory technology, CD-ROM, digital versatile disks (“DVD”) or other optical storage, magnetic cassettes, magnetic tape, magnetic disk storage or other magnetic storage devices, or any other medium which can be used to store the desired information and which can be accessed by a computing device.

Communication methods provide another type of computer readable media; namely communication media. Communication media typically embodies computer-readable instructions, data structures, program modules, or other data in a modulated data signal such as a carrier wave, data signal, or other transport mechanism and include any information delivery media. The terms “modulated data signal,” and “carrier-wave signal” includes a signal that has one or more of its characteristics set or changed in such a manner as to encode information, instructions, data, and the like, in the signal. By way of example, communication media includes wired media such as twisted pair, coaxial cable, fiber optics, wave guides, and other wired media and wireless media such as acoustic, RF, infrared, and other wireless media.

The display 156 can be any suitable display device, such as a monitor, screen, or the like, and can include a printer. In some embodiments, the display is optional. In some embodiments, the display 156 may be integrated into a single unit with the computing device 150, such as a tablet, smart phone, or smart watch. In at least some embodiments, the display is not local to the user. The input device 158 can be, for example, a keyboard, mouse, touch screen, track ball, joystick, voice recognition system, or any combination thereof, or the like. In at least some embodiments, the input device is not local to the user.

The magnetic field generator(s) 162 can be, for example, Helmholtz coils, solenoid coils, planar coils, saddle coils, electromagnets, permanent magnets, or any other suitable arrangement for generating a magnetic field. As an example, the magnetic field generator 162 can include three orthogonal sets of coils to generate magnetic fields along three orthogonal axes. Other coil arrangement can also be used. The optional sensor(s) 164 can include, but are not limited to, one or more position sensors, orientation sensors, accelerometers, image recorders, or the like or any combination thereof.

The one or more magnetometers 160 can be any suitable magnetometer including, but not limited to, any suitable optically pumped magnetometer. Arrays of magnetometers are described in more detail herein. In at least some embodiments, at least one of the one or more magnetometers (or all of the magnetometers) of the system is arranged for operation in the SERF mode.

FIG. 1B is a schematic block diagram of one embodiment of a magnetometer 160 which includes a vapor cell 170 (also referred to as a “cell”) such as an alkali metal vapor cell; a heating device 176 to heat the cell 170; a light source 172; and a detector 174. In addition, coils of a magnetic field generator 162 can be positioned around the vapor cell 170. The vapor cell 170 can include, for example, an alkali metal vapor (for example, rubidium in natural abundance, isotopically enriched rubidium, potassium, or cesium, or any other suitable alkali metal such as lithium, sodium, or francium) and, optionally, one, or both, of a quenching gas (for example, nitrogen) and a buffer gas (for example, nitrogen, helium, neon, or argon). In some embodiments, the vapor cell may include the alkali metal atoms in a prevaporized form prior to heating to generate the vapor.

The light source 172 can include, for example, a laser to, respectively, optically pump the alkali metal atoms and probe the vapor cell. The light source 172 may also include optics (such as lenses, waveplates, collimators, polarizers, and objects with reflective surfaces) for beam shaping and polarization control and for directing the light from the light source to the vapor cell 170 and detector 174. Examples of suitable light sources include, but are not limited to, a diode laser (such as a vertical-cavity surface-emitting laser (VCSEL), distributed Bragg reflector laser (DBR), or distributed feedback laser (DFB)), light-emitting diode (LED), lamp, or any other suitable light source. In some embodiments, the light source 172 may include two light sources: a pump light source and a probe light source.

The detector 174 can include, for example, an optical detector to measure the optical properties of the transmitted probe light field amplitude, phase, or polarization, as quantified through optical absorption and dispersion curves, spectrum, or polarization or the like or any combination thereof. Examples of suitable detectors include, but are not limited to, a photodiode, charge coupled device (CCD) array, CMOS array, camera, photodiode array, single photon avalanche diode (SPAD) array, avalanche photodiode (APD) array, or any other suitable optical sensor array that can measure the change in transmitted light at the optical wavelengths of interest.

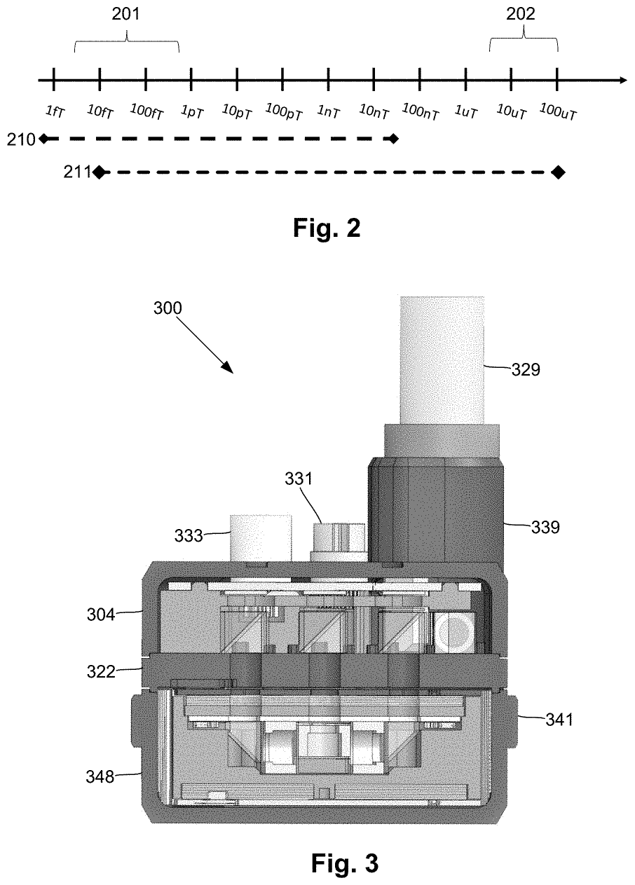

FIG. 2 shows the magnetic spectrum from 1 fT to 100 μT in magnetic field strength on a logarithmic scale. The magnitude of magnetic fields generated by the human brain are indicated by range 201 and the magnitude of the ambient background magnetic field, including the Earth's magnetic field, by range 202. The strength of the Earth's magnetic field covers a range as it depends on the position on the Earth as well as the materials of the surrounding environment where the magnetic field is measured.

Range 210 indicates the approximate measurement range of a magnetometer (e.g., an OPM) operating in the SERF mode (e.g., a SERF magnetometer) and range 211 indicates the approximate measurement range of a magnetometer operating in a scalar mode (e.g., a scalar magnetometer.) Typically, a SERF magnetometer is more sensitive than a scalar magnetometer, but many conventional SERF magnetometers typically only operate up to about 0 to 200 nT while the scalar magnetometer starts in the 10 to 100 fT range but extends above 10 to 100 μT.

An OPM module can include, for example, one or more vapor cells 170 in an optional vapor cell block, one or more detectors 174 in a detector arrangement, a heater 176 in a heating arrangement, and one or more magnetic field generators 162 to provide active shielding in a shielding arrangement. In at least some embodiments, the light source 172 is external to the OPM module and can be provided to a light input of the OPM module.

In at least some embodiments, an OPM module can have a compact arrangement of OPM module components. In at least some embodiments, an OPM module can have a linear arrangement of OPM module components. In at least some embodiments, an OPM module can include various alignment mechanisms for the components.

FIG. 3 illustrates one embodiment of an OPM module 300 having a module case 339 with a housing 322, a cover 304, and a bottom cap 348. In at least some embodiments, the OPM module 300 has a total volume of no more than 50, 40, 30, or 25 cm³ or less. In at least some embodiments, the OPM module 300 has an interior volume of no more than 40, 30, 25, 20, or 18 cm³ or less. In at least some embodiments, the OPM module 300 has an outside diameter of no more than 50, 40, or 35 mm. In at least some embodiments, the OPM module 300 has a height (except for the collimator housing) of no more than 40, 30, or 25 mm and has a total height (with the collimator housing) of no more than 60, 55, or 50 mm.

At least some of the elements of the OPM module 300, systems which can employ the OPM module 300, and methods of making and using the system or OPM modules have been disclosed in U.S. Patent Application Publications Nos. 2020/0072916; 2020/0056263; 2020/0025844; 2020/0057116; 2019/0391213; 2020/0088811; 2020/0057115; 2020/0109481; 2020/0123416; 2020/0191883; 2020/0241094; 2020/0256929; 2020/0309873; 2020/0334559; 2020/0341081; 2020/0381128; 2020/0400763; US 2021/0011094; 2021/0015385; 2021/0041512; 2021/0041513; and 2021/0063510; U.S. patent applications Ser. No. 17/087,988, and U.S. Provisional Patent Applications Ser. Nos. 62/689,696; 62/699,596; 62/719,471; 62/719,475; 62/719,928; 62/723,933; 62/732,327; 62/732,791; 62/741,777; 62/743,343; 62/747,924; 62/745,144; 62/752,067; 62/776,895; 62/781,418; 62/796,958; 62/798,209; 62/798,330; 62/804,539; 62/826,045; 62/827,390; 62/836,421; 62/837,574; 62/837,587; 62/842,818; 62/855,820; 62/858,636; 62/860,001; 62/865,049; 62/873,694; 62/874,887; 62/883,399; 62/883,406; 62/888,858; 62/895,197; 62/896,929; 62/898,461; 62/910,248; 62/913,000; 62/926,032; 62/926,043; 62/933,085; 62/960,548; 62/971,132; 62/983,406; 63/031,469; 63/052,327; 63/076,015; 63/076,880; 63/080,248; 63/089,456; 63/135,364; 63/136,093; 63/136,415; and 63/140,150, all of which are incorporated herein by reference in their entireties.

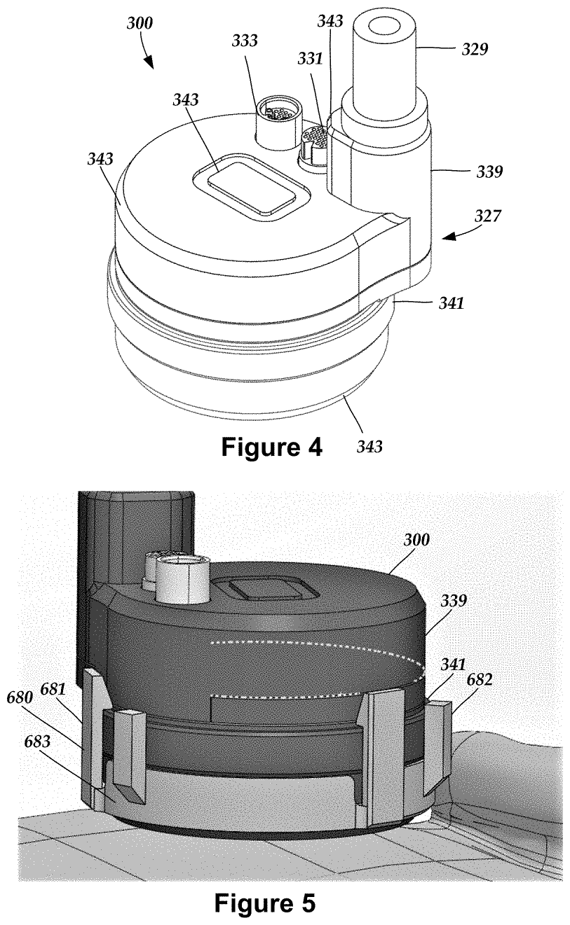

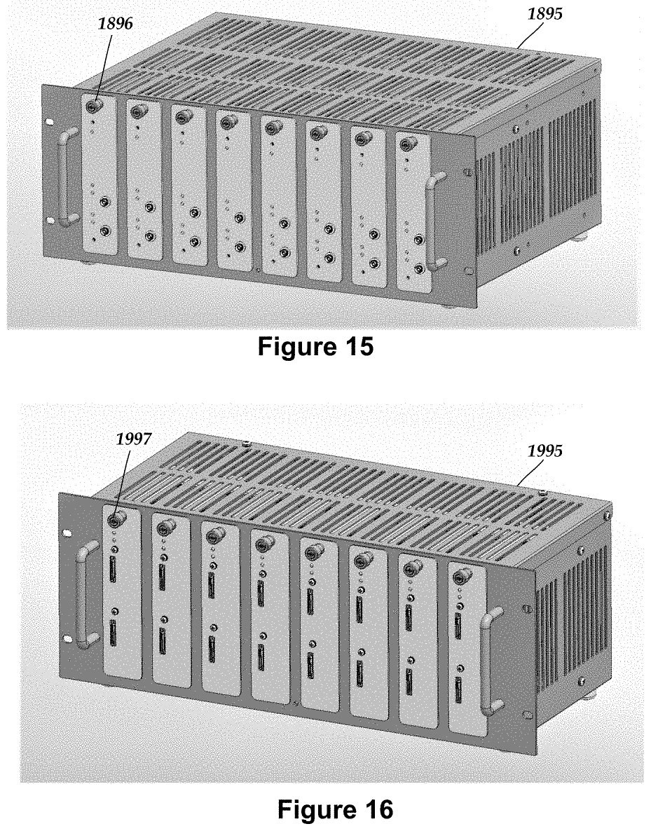

FIG. 4 is an exterior top view of the OPM module 300 with three input/output arrangements 329, 331, 333. From right to left these are the laser input 329, the detector output 331, and the heater/active shielding control input 333. The input/output arrangements 329, 331, 333 provide coupling of the OPM module 300 to components of the magnetic field measurement system. For example, the laser input can be coupled to a light source 172 (FIG. 1B) or a laser module 1895 (FIG. 15) using, for example, optical fibers or other light transmission media. The detector output can be coupled to a sensor module 1995 (FIG. 16) or a computing device 150 (FIG. 1A). The heater/active shielding control can be coupled to a computing device 150 (FIG. 1A) or other controller.

In the illustrated embodiment, the laser output 329 forms a protrusion 327 along the exterior of the module case 339. In at least some embodiments, this protrusion 327 provides an asymmetry for orientation of the OPM module when placed in an assembly, as described below. The case 339 includes a ring 341 extending away from the remainder of the case which can be used to fasten the OPM module in the assembly, as described below. Examples of ornamental features are identified by reference numeral 343 in FIG. 4, for example smooth rounded corners or beveled edges.

In operation, the vapor cells 170 (FIG. 1B) are heated to a desired temperature, but the heater 176 (FIG. 1B) is positioned so that it does not block the laser beams from interrogating the vapor cells. In at least some embodiments, the OPM module can have a heater arrangement that utilizes relatively small resistive elements on a flex circuit substrate to heat the vapor cells. The heater arrangement can also use a ceramic substrate to distribute heat more evenly over the vapor cell array.

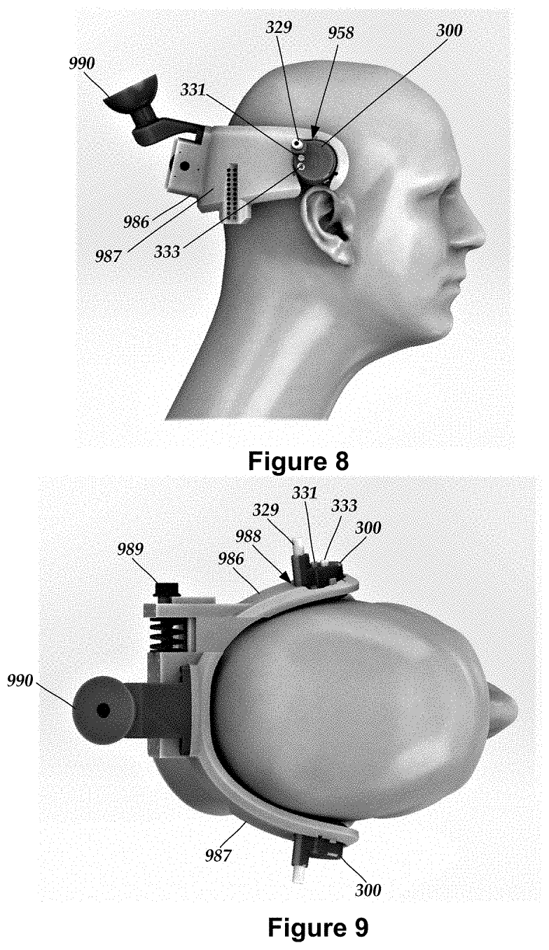

FIG. 5 illustrates one embodiment of a sleeve 680 that fits around the OPM module 300 to fit in a slot in a headgear or other assembly of OPM modules 300 (see, for example, FIGS. 8 through 12, showing exemplary headgear assemblies). In at least some embodiments, the sleeve 680 can facilitate insertion of an OPM module 300 into the headgear or other assembly in a consistent and reliable manner or to a selected or consistent depth or in a specific orientation or any combination thereof. The sleeve 680 includes a number of snap fixtures 681 that snap over the ring 341 on the case 339 of the OPM module 300 to hold the module in the headgear or other assembly in a fixed position. The sleeve 680 may include other fitting features 682 which, in at least some embodiments and alone or with the snap fixtures 681, may be used to position the OPM module on the headgear or other assembly in a specific orientation. In at least some embodiments, the sleeve 680 in general or one or more of the snap fixtures 681 or other fitting fixtures 682 may limit the depth that the OPM module 300 can be inserted into the headgear or other assembly. In at least some embodiments, the sleeve 680 is part of the module support (for example, module support 987 of FIGS. 8 and 9) of the headgear or other assembly. In at least some embodiments, the sleeve 680 can be a separate element that fits into ports 988 (FIGS. 8 and 9) of the module support 987.

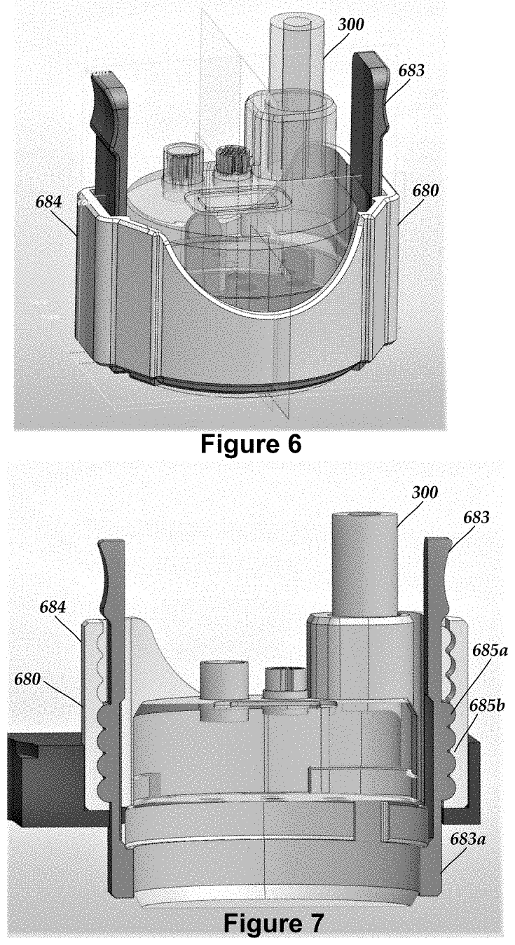

FIGS. 6 and 7 illustrate another embodiment of a sleeve 680. This embodiment includes an inner sleeve 683 and an outer sleeve 684. Each of the inner sleeve 683 and the outer sleeve 684 includes a set of complementary repeating detent features 685a, 685b. This allows adjustment, for a particular user's head, of the depth that the OPM module 300 may extend into the headgear or other assembly by sliding the inner sleeve 683 further into or out of the outer sleeve 684 using the repeating detent features 685a, 685b to define multiple different depths. The sleeves 680 of FIGS. 5 to 7 include a flange 683a that fits under the ring 341 of the OPM module 300.

Knowledge of the location of the OPM module 300 relative to the head of the user is important for neural signal measurement and identification. The sleeves 680 of FIGS. 5 through 7 provide adjustment mechanisms for reliably determining the depth (and, in at least some embodiments, the orientation) of the OPM module 300 in the headgear or other assembly. The sleeves 680 can be made of any suitable material including plastic materials and can be flexible or rigid.

A variety of different headgears or other assemblies can be constructed using the OPM modules 300 (optionally disposed in sleeves 680). In at least some embodiments, the system can have a headgear with two module supports and an adjustment mechanism used for adjusting the headgear to conform with the width of the user's head. In at least some embodiments, the system can have a helmet with at least 256 vapor cells.

FIGS. 8 and 9 illustrate one embodiment of a headgear 986 with two module supports 987 disposed on opposite sides of the head of the user with each module support receiving one OPM module 300 in a port 988 in the module support. In at least some embodiments, the ports 988 can be constructed so that the OPM module 300 or sleeve 680 can be inserted only at one or more specific orientations. When an OPM module 300 is inserted into the port 988 in the module support 986, the input/output arrangements 329, 331, 333 remain exposed so that they can be coupled to corresponding components of the magnetic field measurement system. The other headgear disclosed below can also have ports for receiving the individual OPM modules 300 similar to the ports 988 of the module support 986. The OPM modules 300 may be positioned by the arrangement of the headgear 986 to record signals from particular parts of the brain, such as, for example, the auditory centers of the left and right hemispheres of the brain.

In at least some embodiments, the headgear 986 includes an adjustment mechanism 989 (shown in FIG. 9) that allows for adjustment of the width of the headgear (i.e., the separation distance between the two module supports 987) to fit users having different head widths. In the illustrated embodiment, the adjustment mechanism includes a threaded bolt that can be screwed in or out to adjust the separation between the two module supports 987. Any other suitable adjustment mechanism can be used.

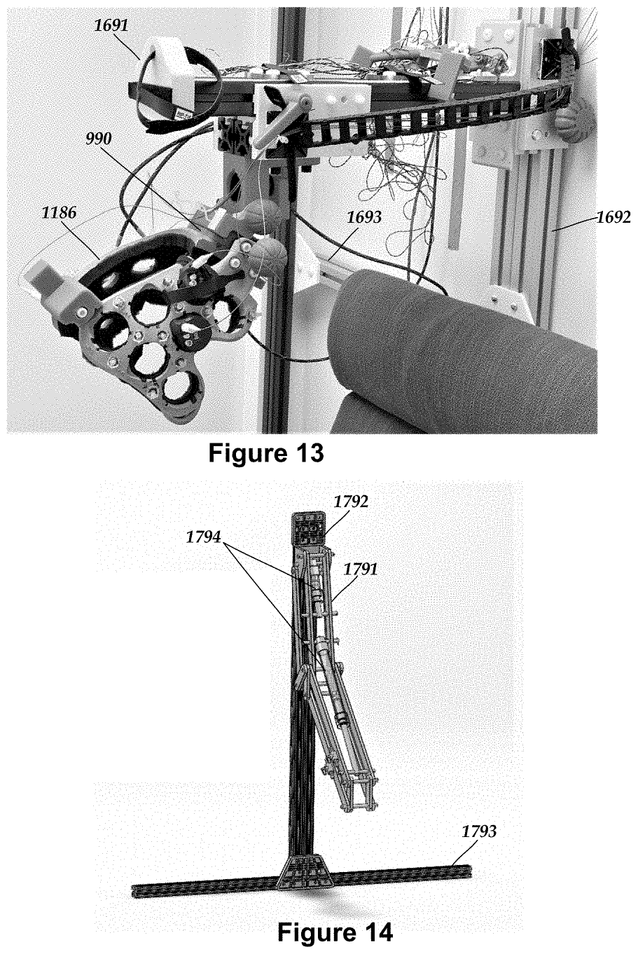

In addition, the headgear 986 includes a connection element 990 that can be coupled to an arm 1691, 1791 (shown in FIGS. 13 and 14), as described in more detail below. Examples of connection elements can include balls, sockets, bolts, screws, or other fasteners or the like or any combination thereof.

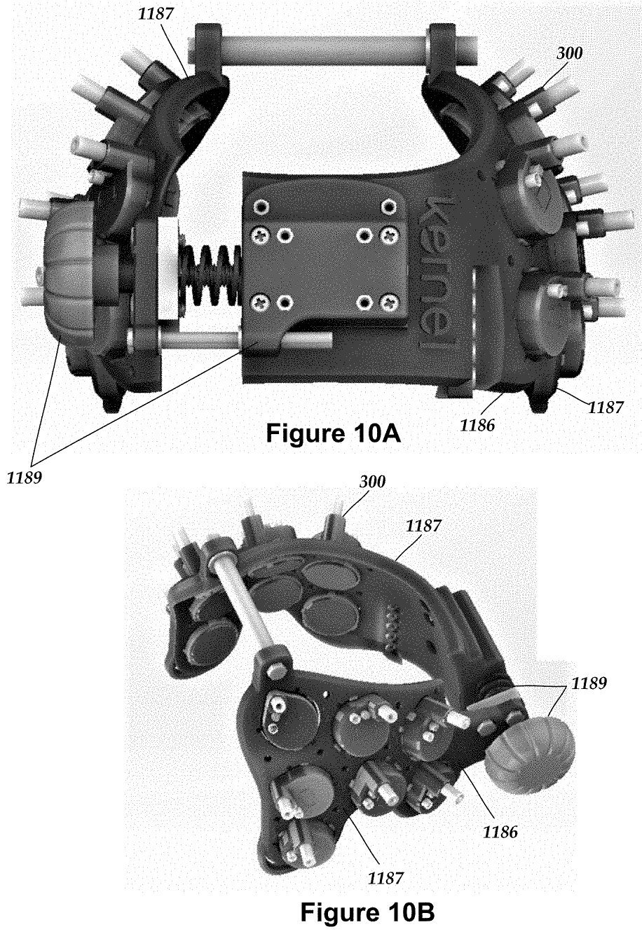

FIGS. 10A and 10B illustrate another embodiment of a headgear 1186 that is configured to receive fourteen OPM modules 300 distributed between two module supports 1187. Again, this headgear 1186 includes an adjustment mechanism 1189 that allows for adjustment of the width of the headgear. Any suitable number of OPM modules 300 can be used in the headgear 986, 1186 described herein including, but not limited to, one, two, three, four, five, six, eight, ten, twelve, fourteen, sixteen, eighteen, twenty, twenty-four, thirty, or more OPM modules. Each OPM module 300 can include any suitable number of vapor cells 170 (FIG. 1B) including, but not limited to, one, two, three, four, five, six, seven, eight, nine, ten, twelve or more vapor cells. The OPM modules 300 can have the same number of vapor cells 170 (FIG. 1B) or different numbers of vapor cells. The headgear 1186 can also include a connection element similar to connection element 990.

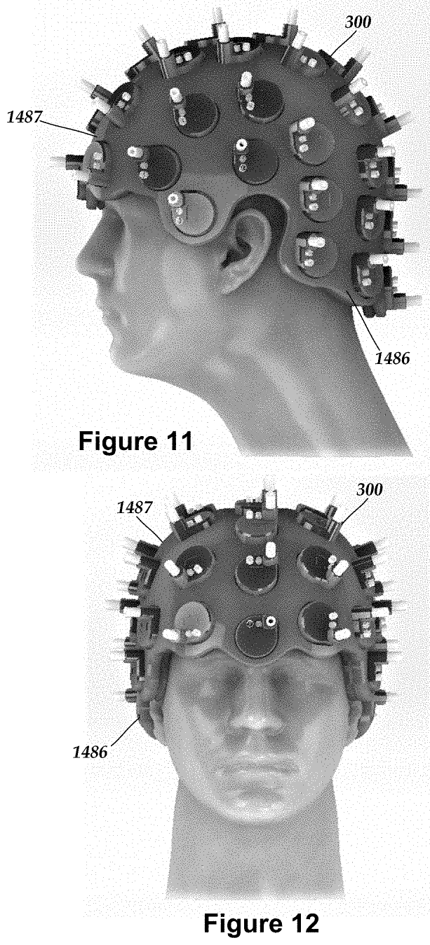

FIGS. 11 and 12 illustrate a further embodiment of a headgear, e.g., a helmet 1486, with OPM modules 300 disposed around the helmet in a module support 1487. The helmet can include any suitable number of OPM modules 300 including, but not limited to, 1, 2, 8, 10, 16, 20, 24, 30, 32, 40, 48, 50, 60, 64, or more OPM modules. Each OPM module 300 can include any suitable number of vapor cells 170 (FIG. 1B) including, but not limited to, one, two, three, four, five, six, seven, eight, nine, ten, twelve or more vapor cells. The OPM modules 300 can have the same number of vapor cells 170 (FIG. 1B) or different numbers of vapor cells. In at least some embodiments, the helmet 1486 includes at least 200, 250, 256, 300, 400, 450, 500, or 512 vapor cells distributed in the OPM modules 300 disposed around the user's head. This can provide sufficient sensor density to capture spatial information near the scalp of the user. The OPM modules 300 can be provided in any uniform or non-uniform arrangement in the helmet 1486. The arrangement of the OPM modules 300 on the left and right hemispheres of the helmet 1486 can be the same or different.

In other embodiments, the helmet 1486 may include one or more adjustment mechanisms to adjust the helmet to fit the user's head. The helmet 1486 can also include a connection element similar to connection element 990.

In at least some embodiments, the system can have an adjustable arm that supports the complete headgear weight, to control head motion within specific degrees of freedom and ranges of motion, and with features for cable/fiber protection and routing. In at least some embodiments, the system can have laser and sensor chassis which allow close placement to the OPM modules, to reduce cable and fiber lengths.

FIG. 13 illustrates an arm 1691 for coupling to connection element 990 of any one of the headgear 986, 1186, 1486 described above. In at least some embodiments, the arm 1691 may be arranged to hold the user's head in a fixed position. In other embodiments, the arm 1691 may permit limited or relatively free movement of the head of the user with the headgear 986, 1186, 1486. In at least some embodiments, the arm 1691 supports the weight of the headgear 986, 1186, 1486, not the user. In at least some embodiments, the arm 1691 attaches to the connection element 990 of the headgear 986, 1186, 1486.

The arm 1691 of FIG. 13 can move vertically along one track 1692 to adjust for height and along another track 1693 for horizontal positioning. In at least some embodiments, the arm 1691 includes one or more ball and socket mounts (not shown) or other joint arrangements to provide additional adjustments.

FIG. 14 illustrates another arrangement of an arm 1791 that uses two tracks 1792, 1793 for vertical and horizontal movement and one or more hydraulic arm segments 1794 to provide additional adjustments. In at least some embodiments, the arm 1791 attaches to the connection element 990 of the headgear 986, 1186, 1486. In at least some embodiments, the arm 1791 supports the weight of the headgear 986, 1186, 1486, not the user.

In at least some embodiments, the arm 1691, 1791; tracks 1692, 1693, 1792, 1793; ball and socket mounts; and hydraulic arm segments 1794 can be made partially or entirely from non-magnetic materials such as, for example, aluminum or polymers.

In at least some embodiments, the system can have laser/sensor modules in a non-magnetic chassis with passive air flow and a heat sink for cooling to avoid the use of fans. In at least some embodiments, the system can have a peripheral made of non-magnetic materials for user input.

FIG. 15 illustrates a chassis 1895 for a set of laser modules 1896 to provide laser beams to the OPM modules 300 through the laser input 329 (FIG. 4). FIG. 16 illustrates a chassis 1995 for a set of sensor modules 1997 to communicate with the detectors 174 (FIG. 1B) through the detector output 331 (FIG. 4). Because the OPM modules 300 are measuring weak magnetic field signals, the reduction of stray magnetic field noise is beneficial. In at least some embodiments, the chassis 1895, 1995 of the laser modules 1896 or sensor modules 1997 and other components can be made using non-magnetic materials, such as aluminum or the like. In at least some embodiments, the laser and sensor modules 1896, 1997 can be air cooled in the chassis 1895, 1995 to avoid the use of fans which generate magnetic field noise and vibrations and can include heat sink for further heat dissipation. These features allow the laser and sensor modules 1896, 1997 to be placed closer to the headgear 986, 1186, 1486 and OPM modules 300.

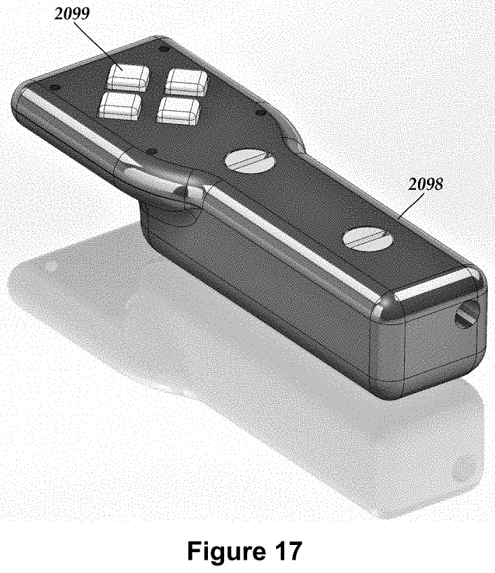

FIG. 17 illustrates a peripheral 2098 (e.g., handheld remote) that can be used by the user to provide user input using buttons 2099 or other input elements (for example, a joystick, trackpad, or the like) on the peripheral. The peripheral is made of non-magnetic materials, such as a plastic enclosure, conductive button contacts, and a printed circuit board (PCB) to cable electrical connection to reduce or prevent magnetic field noise.

The above specification provides a description of the invention and its manufacture and use. Since many embodiments of the invention can be made without departing from the spirit and scope of the invention, the invention also resides in the claims hereinafter appended.

What is claimed as new and desired to be protected by Letters Patent of the United States is:

1. A headgear for magnetoencephalography, the headgear comprising:
a body defining a plurality of ports, wherein the body comprises a first portion and a second portion;
an adjustment mechanism coupled to the first portion and the second portion of the body and configured to adjust a separation between the first and second portion to facilitate fitting the headgear to a head of a user; and
a plurality of optically pumped magnetometer (OPM) modules, wherein each of the OPM modules comprises at least one vapor cell and is configured to be removably inserted into a one of the ports of the body, wherein each of the OPM modules is configured for coupling to a light source for receiving light.

2. The headgear of claim 1, wherein the plurality of OPM modules is at least two OPM modules with at least one of the OPM modules disposed in the first section and at least another one of the OPM modules disposed in the second section.

3. The headgear of claim 2, wherein the plurality of OPM modules is at least ten OPM modules.

4. The headgear of claim 1, further comprising a connection element coupled to the body for coupling the headgear to an arm.

5. A magnetic field measurement system, comprising
the headgear of claim 1; and
at least one laser module coupleable to the OPM modules to provide the laser light.

6. The magnetic field measurement system of claim 5, further comprising at least one sensor module coupleable to the OPM modules to receive output from the detector arrangement.

7. The magnetic field measurement system of claim 6, further comprising at least one peripheral configured for input by a user of the magnetic field measurement system while the user wears the headgear.

8. A headgear for magnetoencephalography, the headgear comprising:
a body defining a plurality of ports; and
a plurality of optically pumped magnetometer (OPM) modules, wherein each of the OPM modules comprises at least one vapor cell and is configured to be removably inserted into a one of the ports of the body, wherein each of the OPM modules is configured for coupling to a light source for receiving light and the OPM modules comprise, in total, at least 200 vapor cells.

9. The headgear of claim 8, wherein the OPM modules comprise, in total, at least 256 vapor cells.

10. The headgear of claim 8, wherein each of the OPM modules comprises at least six vapor cells.

11. A magnetic field measurement system, comprising
the headgear of claim 8; and
at least one laser module coupleable to the OPM modules to provide the laser light.

12. The magnetic field measurement system of claim 11, further comprising at least one sensor module coupleable to the OPM modules to receive output from the detector arrangement.

13. The magnetic field measurement system of claim 12, further comprising at least one peripheral configured for input by a user of the magnetic field measurement system while the user wears the headgear.

14. A headgear for magnetoencephalography, the headgear comprising:
a body defining a plurality of ports;
a plurality of optically pumped magnetometer (OPM) modules, wherein each of the OPM modules comprises at least one vapor cell and is configured to be removably inserted into a one of the ports of the body, wherein each of the OPM modules is configured for coupling to a light source for receiving light; and
a plurality of sleeves, wherein each of the sleeves is configured to fit around a one of the OPM modules and configured to fit, with the OPM module, in a one of the ports and to fix the OPM module within the one of the ports.

15. The headgear of claim 14, wherein each of the sleeves comprises at least one snap fit fixture configured to snap fit over a feature on the OMP module.

16. The headgear of claim 14, wherein each of the sleeves comprises an inner sleeve and an outer sleeve that is movable relative to the inner sleeve.

17. The headgear of claim 16, wherein the inner sleeve and the outer sleeve have complementary sets of detents that facilitate positioning the inner sleeve at different depths relative to the outer sleeve by matching the sets of detents.

18. A magnetic field measurement system, comprising
the headgear of claim 14; and
at least one laser module coupleable to the OPM modules to provide the laser light.

19. The magnetic field measurement system of claim 18, further comprising at least one sensor module coupleable to the OPM modules to receive output from the detector arrangement.

20. The magnetic field measurement system of claim 19, further comprising at least one peripheral configured for input by a user of the magnetic field measurement system while the user wears the headgear.
